(19) United States
(12) Patent Application Publication
(10) Pub. No.: US 2009/0143588 A1
(43) Pub. Date: Jun. 4, 2009

(54) QUARTZ GLASS MICRO-PHOTOREACTOR AND SYNTHESIS OF 10-HYDROXYCAMPTOTHECIN AND 7-ALKYL 10-HYDROXYCAMPTOTHECIN

(75) Inventors: Silvia WERNER, Kahl (DE); Holger Rauter, Flieden (DE); Friedrich Wissmann, Alzenau (DE); Raimund Seliger, Freigericht (DE)

Correspondence Address: NORRIS, MCLAUGHLIN & MARCUS, PA, 875 THIRD AVENUE, 18TH FLOOR, NEW YORK, NY, 10022, US

(73) Assignee: W.C. HERAEUS GMBH, Hanau (DE)

(21) Appl. No.: 12/277,854

(22) Filed: Nov. 25, 2008

(30) Foreign Application Priority Data
Nov. 29, 2007 (DE) 10 2007 057 869.7

(51) Int. Cl. C07D 491/22; B01J 19/08

(52) U.S. Cl. 546/48; 422/186

(57) ABSTRACT

A process for the production of 10-hydroxycamptothecin

hydroxycamptothecin and/or of 7-alkyl 10-hydroxycamptothecin, with the steps of
A Providing a continuous fluid stream with a thickness d of a solution of camptothecin N-oxide or 7-alkyl camptothecin N-oxide; and
B. Passing the stream past at least one source of light which produces radiation parallel to the thickness d, d being 10 μm to 1000 μm;
   and devices for executing the process.

The invention relates to processes for the production of 10-hydroxycamptothecin and of 7-alkyl 10-hydroxycamptothecin and to devices for executing the processes.

### BACKGROUND

EP 0 074 256 A 1 (“Camptothecin derivatives, process for preparing same, formulations containing such derivatives and their use”) and U.S. Pat. No. 4,545,880 (“Photochemical process for preparing Camptothecin derivatives”, example 7) describe the production of 10-hydroxycamptothecin and 7-alkyl 10-hydroxycamptothecin by irradiating camptothecin N-oxide or 7-alkyl camptothecin N-oxide in a solvent, preferably dioxane, acetonitrile, chloroform, dichloromethane, glyme, diglyme and mixtures thereof with an addition of water in the presence of an acid, preferably mineral acids, organic sulfonic acids, Lewis acids and organic carboxylic acids. The irradiation of the reaction mixture with UV light takes place in accordance with the state of the art known at the time using a mercury lamp.

In EP 0 074 256 A1 it is described that the reaction of the N-oxides leads to two different reaction products, namely the starting material deoxygenated in position 1 (camptothecin or 7-alkyl camptothecin) and the desired 10-hydroxy derivatives. 0-Hydroxycamptothecin was obtained from camptothecin N-oxide by photolysis in dioxane/MeCN/H₂O (5:4:1) in the presence of H₂SO₄ in a yield of 88.2%.

Lei et al., Chemical Research in Chinese Universities, 17 (1), 69-72 (2001), describe a three-stage process, starting out from camptothecin (CAS 7689-03-4), which process leads to 7-ethyl-10-hydroxycamptothecin (SN-38), irradiation with an Hg high pressure lamp being carried out in the last photochemical stage.

JP2006290765A (“Method for the production of 7-alkyl 10-hydroxycamptothecins”) relates to the synthesis of 7-alkyl-10-hydroxycamptothecins from 7-alkyl camptothecin N-oxides, irradiation with UV light being carried out in the presence of acids. The reaction time is indicated as being 100 min and the yield as being 79.2%. The formation of the by-products (camptothecin or 7-alkyl camptothecin) and consequently a reduced yield are discussed and the use of UV radiation the wave length range of which is such that no wavelengths shorter than 370 nm are emitted is proposed as the solution for the yield reduction. In a preferred embodiment, irradiation with light in the range of 370 to 480 nm (low pressure mercury lamp 370-480 nm, metal halide lamp 400-430 nm, light emitting diode 405 nm, laser diode 405 nm) is carried out.

By using the radiation spectrum of 370 to 480 nm, it was possible to reduce the content of 7-ethyl camptothecin according to JP 2006290765 from approximately 12% to as low as 3.8%. It was possible to achieve an increase in yield from approximately 50% to 80% by using a tubular flow reactor instead of a batch reactor.

The object arises of providing a process which is suitable for the industrial production of 10-hydroxycamptothecin and 7-alkyl 10-hydroxycamptothecins. In addition, it is an object of the present invention to develop a suitable reactor for the photochemical production of camptothecin derivatives, which reactor permits the production of such products and similar compounds under reproducible, economic conditions simple for the user on an industrial scale.

### Detailed Description: Process

The invention relates to processes for the production of 10-hydroxycamptothecin and 7-alkyl-10-hydroxycamptothecin with the steps of
A. Providing a continuous fluid stream—which is preferably cuboid and/or irradiated on both sides—with a thickness d of a solution of camptothecin N-oxide and/or 7-alkyl camptothecin N-oxide;
B. Passing the fluid stream past sources of light which produce radiation parallel to the thickness d, d being 20 μm to 1000 μm.

Preferably, d is 40 μm to 100 μm. Advantageously, the fluid stream is cuboid (length 1, width b, thickness d). Preferably, irradiation is carried out from both sides parallel to the thickness d. In an equally preferred embodiment, irradiation with light in the range of 350 to 400 nm is carried out.

Suitable sources of light for this purpose are e.g. low pressure mercury lamps with suitable spectral filters (350-400 nm), LEDs (light emitting diodes (375 nm or 385 nm)).

According to the present invention, it is possible to greatly reduce the content of 7-alkyl camptothecin by-products by using a plane flow geometry irradiated from both sides and LED sources of radiation with a wavelength of 385 nm or Hg high pressure emitters with spectral filters (350 nm to 400 nm).

In addition, it is possible according to the present invention, to increase the concentration of the solutions, to be irradiated, of 7-alkyl camptothecin N-oxide from the previously described 0.1% to as much as 3% and more by using dimethyl formamide (DMF) or DMF/dioxane as solvent.

In addition, it has been found that the range of by-products can be substantially improved by carrying out the irradiation with a tempered fluid stream of camptothecin N-oxide or 7-alkyl camptothecin N-oxide. In the preferred case, the temperature of the fluid stream is 40-80° C.

In addition, it has been found that, if irradiated from both sides, solutions of camptothecin N-oxide and 7-alkyl camptothecin N-oxide with an increased concentration can still be irradiated so completely in the case of a greater irradiated layer thickness d that a conversion rate of more than 99% can be achieved with a high flow rate.

The following examples illustrate the process in further detail. As in the rest of the description, parts and percentages relate to the weight, unless indicated otherwise.

### COMPARATIVE EXAMPLE 1

Photochemical irradiation of an 0.1% solution of 7-ethyl camptothecin N-oxide for the production of 7-ethyl-10-hydroxycamptothecin in a photoreactor (falling film photoreactor according to Dr. de Meijere; storage vessel 1000 ml) with an Hg high pressure emitter designed as immersion lamp (Heraeus Noblelight, type TQ 718 Z 2; approximately 10% of the total output is emitted in the range of 350 to 400 nm).

7-Ethyl camptothecin N-oxide (0.75 g, 1.91 mmole) is dissolved in a mixture of 1,4-dioxane (730 ml) and 1 N sulfuric acid (1.75 ml) and irradiated in a photoreactor with an Hg immersion lamp for 30 min. The irradiated solution is subsequently concentrated under vacuum and suspended in t-butyl methyl ethyl ether (10 ml) with stirring, filtered and washed further with diisopropyl ether (10 ml). The product is dried at 50° C. under vacuum. The crude product contaminated with 11% 7-ethyl camptothecin is purified by column chromatography (silica gel; chloroform:methanol 90:10). Cleaned fractions are concentrated, filtered and additionally washed with t-butyl methyl ether (10 ml). The product is dried under vacuum at 50° C. Yield: 0.37 g (49%).

### EXAMPLE 1

Photochemical irradiation of an 0.3% solution of 7-ethyl camptothecin N-oxide for the production of 7-ethyl-10-hydroxycamptothecin in a micro-photoreactor type A with high performance LED array (Epitex; type L 385-30 with a typical emission wavelength of 285 nm and an irradiation performance of 150 mW).

7-Ethyl camptothecin N-oxide (150 g, 0.382 mole) is dissolved in a mixture of 1,4-dioxane (46 l), N,N-dimethyl formamide (2.7 l), water (38 g) and concentrated sulfuric acid (38 g) and conveyed through two micro-photoreactors of type A arranged in series at a flow rate of 600 ml/h and at a fluid temperature of 70-75° C. The irradiated solution is subsequently concentrated to 200 ml under vacuum. The mixture obtained is then introduced into water (6.3 l) at 15 to 30° C. with stirring and stirred further. The product is filtered, washed three times with water (1.5 l) and dried in a stream of nitrogen at 50° C. Content of 7-ethyl camptothecin 2.8%. Yield: 127 g (85%).

### EXAMPLE 2

Photochemical irradiation of an 0.6% solution of 7-ethyl camptothecin N-oxide for the production of 7-ethyl-10-hydroxycamptothecin in a quartz glass micro-photoreactor type B with Hg high pressure emitters (Heraeus Noblelight, medical UV unit) and interference filters with specified band pass and a coating on both sides; UV emission wavelength range 350 nm to 400 nm.

7-Ethyl camptothecin N-oxide (150 g, 0.382 mole) is dissolved in a mixture of 1,4-dioxane (23.1 l), N,N-dimethyl formamide (1.4 l), water (38 g) and concentrated sulfuric acid (38 g) and conveyed through a quartz glass micro-photoreactor type B at a flow rate of 900 ml/h and at a fluid temperature of 70-75° C. The irradiated solution is subsequently concentrated to 200 ml under vacuum. The mixture obtained is then introduced into water (6.3 l) with stirring. The product is filtered, washed three times with water (1.3 l) and dried in a stream of nitrogen at 50° C. Yield: 143 g (95%).

### EXAMPLE 3

Photochemical irradiation of an 0.25% solution of camptothecin N-oxide for the production of 10-hydroxycamptothecin in a micro-photoreactor type A with high performance LED arrays (Epitex, type L 385-30, typical emission wavelengths 385 nm and irradiation performance 150 mW).

Camptothecin N-oxide (2.5 g, 0.004 mole) is dissolved in a mixture of 1,4-dioxane (833 ml), N,N-dimethyl formamide (167 ml), water (2.5 ml) and concentrated sulfuric acid (1.5 ml) at 550° C. and conveyed through two micro-photoreactors of type A arranged in series at a flow rate of 480 ml/h and at a fluid temperature of 70-75° C. The irradiated solution is subsequently concentrated under vacuum and introduced into water (200 ml) with stirring. The product is filtered, washed four times with water (25 ml in each case) and dried at 40° C. under vacuum. Content of camptothecin 4.3% (the starting material contains approximately 1% camptothecin). Yield: 1.75 g (70%).

### EXAMPLE 4

Photochemical irradiation of an 0.45% solution of camptothecin N-oxide for the production of 10-hydroxycamptothecin in a quartz glass micro-photoreactor type B Hg high pressure emitters (Heraeus Noblelight) and interference filters with specific band pass and coating on both sides; UV emission wavelengths range 350 nm to 400 nm.

Camptothecin N-oxide (4.5 g, 0.012 mole) is dissolved in a mixture of 1,4-dioxane (833 ml), N,N-dimethyl formamide (167 ml), water (4.5 ml) and concentrated sulfuric acid (2.7 ml) at 50° C. and conveyed through a quartz glass micro-photoreactor type B at a flow rate of 720 ml/h and a fluid temperature of 70-75° C. The irradiated solution is subsequently concentrated under vacuum and introduced into water (500 ml) with stirring. The product is filtered, washed with water and dried at 40° C. under vacuum. Yield: 4.2 g (93%).

### Detailed Description: Equipment

Surprisingly enough, it has been found that the flow behavior of a cuboid quartz glass flow cell greatly enlarged as regards its dimensions (compare FIG. 1) permits a sufficiently homogenous flow with accurately adjustable residence time behavior of the solution. Consequently, a flow cell designed in this way is suitable as a photoreactor simple to produce.

A device suitable according to the invention consists of a design of a micro-photoreactor improved in terms of its characteristics in the form of a glass flow cuvette (quartz glass micro-photoreactor type B) with a minimized gap width, which is produced in such a way that is consists of two firmly connected, transparent, level discs. These discs connected at a defined distance from each other form a gap through which the solution to be irradiated can be transported via a feed and discharge facility. The glass cuvette can be irradiated from outside by means of sources of light emitting light of a suitable wavelength. The source of light can be integrated into an irradiation unit in such a way that filter discs are used which filter out undesirable wavelength ranges.

In contrast to known micro reactors, the transparent plates of the quartz glass micro-photoreactor according to the invention are connected with each other not in a solidly locked or form-matching but in a firmly bonded manner. This has the major advantage that it permits adjusting a defined gap width more reproducibly and precisely.

If two plates are connected in a solidly locked or form-matching manner, the utilization of two transparent parts consisting of glass is not preferred or disadvantageous. When adjusting a narrowly defined gap width, a force needs to be applied vertically to the plates in each case, which force is sufficient to reach the gap dimension. As a result of this force effect, the glass parts can become damaged. In this case, elastic materials need to be used between the glass parts which materials clearly make an accurate adjustment of the gap width difficult.

A firmly bonded connection, on the other hand, permits a pressure-free setting of a gap by the corresponding connection between two level plates and bridges situated in-between.

In DE 103 41 500 A1, the depth of penetration and consequently the gap width of a photoreactor is derived from the Lambert-Beer law. A quartz glass micro-photoreactor irradiated from two sides offers, for obvious reasons, a depth of penetration of the light into the medium to be irradiated, which is twice as large; for this reason, it is possible to operate with larger gap widths easier to produce. On the other hand, it is possible to operate also with solutions of a higher educt concentration with the same gap width, leading to marked cost savings in terms of solvent.

Typical gap widths of the quartz glass micro-photoreactor described here and produced from a quartz glass flow cuvette amount to 20 μm to 1000 μm, preferred gap widths amount to 40 μm to 100 μm.

An important component of a micro-photoreactor is its source of light and its arrangement in terms of design in the system. In order to eliminate a mutual negative influence of the reaction medium and the light supply respectively as far as possible, the arrangement of the source of light in a spatially separated irradiation unit is of advantage.

A quartz glass micro-photoreactor type B with Hg high pressure emitters (Heraeus Noblelight) and spectral filters with a specific band pass and a coating on both sides; UV emission wavelength range 350 nm to 400 nm; output 500 W has proved suitable. The Hg emitter may also be coated with metal halide ions.

Using a single such reactor, approximately 130 g per day of 7-ethyl camptothecin N-oxide can be reacted. By using reactor systems operating correspondingly in parallel (numbering up) quantities in the kg range can be produced on an industrial scale daily. It is also possible to connect two reactors behind each other in series.

Micro-reactors, just as the quartz glass micro-photoreactor described here, can become blocked if educts, intermediates or end products are crystallized out from the reaction medium or separate out amorphously. This leads to leakages or even bursting of the reactors. As a result, reaction medium or substances dissolved therein come into contact with the sources of light with the possibility of sources of light fitted directly to the reactor becoming damaged.

According to the invention, the sources of radiation are therefore preferably housed in a separate, closed radiation unit which can be installed at a defined distance and angle from the reactor surface to be irradiated.

A preferred embodiment of such a spatially separated radiation unit consists of a housing of suitable construction material, e.g. a metallic material, in a suitable form, e.g. cuboid, cylindrical, conical or similar, which encloses the source of radiation on all sides externally towards the reactor. This radiation unit is closed off by a transparent plate facing the reactor side.

In a further preferred embodiment, this transparent plate can be executed as spectral filter such that only light of the desired wavelength in the UV, visible light and/or IR range is emitted onto the reactor.

In a further embodiment, the spectral filter can be fitted directly onto the glass panes of the micro-photoreactor in order to achieve the desired wavelength in the UV, visible light and/or IR range.

Light in the IR range, in particular, may be useful if heating of the photoreactor and the medium flowing through is to be achieved simultaneously by means of the source of light.

Another possibility for heating the reaction medium consists of applying a thin layer of indium tin oxide or other coating materials with transparent conductive properties onto the external surface of the quartz glass micro-photoreactor. Into each irradiation unit, at least one source of radiation is installed. The number of radiation sources depends on the necessary radiation output for the photoreaction to be carried out and on whether different sources with different wavelength emissions are advantageous.

In a preferred embodiment, gas discharge lamps or semiconductor sources of light are used.

### Description of the Photoreactors Tested

Photoreactor with an Hg Immersion Lamp

The photoreactor (falling film photoreactor according to Dr. de Meijere) consists of an irradiation vessel (volume 200 ml) with a silver mirrored high vacuum jacket with sight strip and warming jacket. In the irradiation vessel, there is the cooling tube of quartz glass and the dip pipe of boron silicate glass. The circulation of the liquid is effected by means of a forced circulation pump, system Normag, which is controlled via a control device. The storage vessel has a volume of 1000 ml. An Hg high pressure lamp TQ 718 Z2 (Heraeus Noblelight), designed as an immersion lamp, with metal halide additive coatings, is used as source of radiation. This Hg emitter emits the characteristic Hg line spectrum, which ranges from the short-wave UV range of a wavelength of approximately 240 nm to far inside the visible region. Within this range, a few intense and several weaker lines are present. The strongest line within the UV range is near the wavelength of 366 nm. The subsequent lines in the visible range of between 400 and 600 nm, too, are frequently effective in photochemical reactions.

### Micro-Photoreactor Type A

The photoreactor consists of the sub-assembly of reactor unit and emitter unit. The reactor unit consists essentially of a solid stainless steel block (235 mm×210 mm×22 mm) and a glass plate (205 mm×150 mm×10 mm) held in a metal frame. The stainless steel block has the purpose of conducting the process fluid. Outside the fluid zone, there is additionally a Dartek® substrate of 20 μm. A glass plate is pressed onto this Dartek® substrate.

Warming of the process fluid solution takes place by a separate current circuit.

The source of radiation consists of an array block of 50 high performance UV LED arrays with a typical emission wavelength of 385 nm and an irradiation performance per high performance UV LED array of 150 MW. The emitter unit is flushed with nitrogen in order to avoid condensation on the partially cooled electrical components on the one hand, the penetration into the sub-assembly of possibly flammable gas mixtures from outside, on the other hand.

The source of radiation is positioned correctly onto the reactor part.

### Quartz Glass Micro-Photoreactor Type B

The quartz glass micro-photoreactor consists of the reactor unit R and two separately closed-off irradiation units (source of radiation).

The reactor unit R consists essentially of two quartz glass plates P1 and P2. The gap depth S is defined by a separator which is also made of quartz glass. The quartz glass plates P1, P2 and the separator are connected flush with each other.

The fluid stream flows at a flow rate v₂ through a bore and can pass through an entry canal before it reaches the irradiated zone.

Each irradiation unit (source of radiation) consists of an Hg high pressure emitter (Heraeus Noblelight) and a spectral filter with a specific band pass and a coating on both sides such that the UV emission wavelength range of 350 nm to 400 nm is reached. The output of the Hg high pressure emitter is 500 W. The Hg emitter can also be coated with metal halide ions.

The IR radiation of the Hg high pressure emitter is used to heat the fluid stream.

### BRIEF DESCRIPTION OF THE DRAWINGS

FIG. 1 shows a quartz glass micro-photoreactor type B according to the invention irradiated on both sides with the reactor unit and two sources of radiation. In the case of the device shown in FIG. 1, the sources of radiation are housed in a separate, closed-off irradiation unit.

FIG. 2 shows the reactor unit R with an entry and exit facility (FIG. 4 6,7) for the fluid stream with a flow rate v₂.

FIG. 3 shows a diagrammatic arrangement of a quartz glass micro-photoreactor irradiated on both sides.

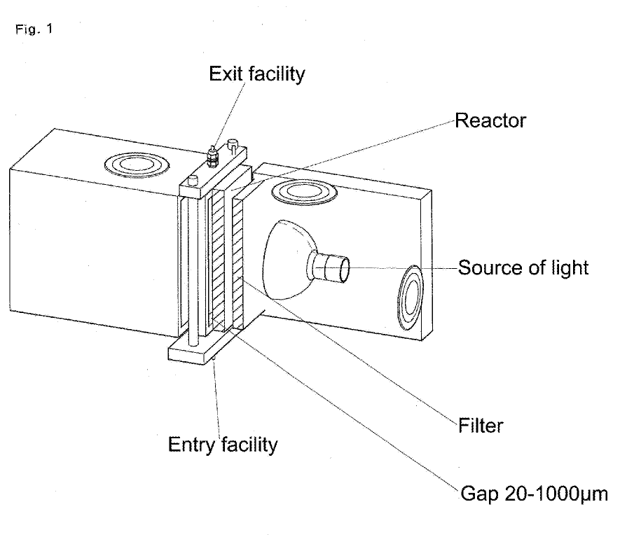

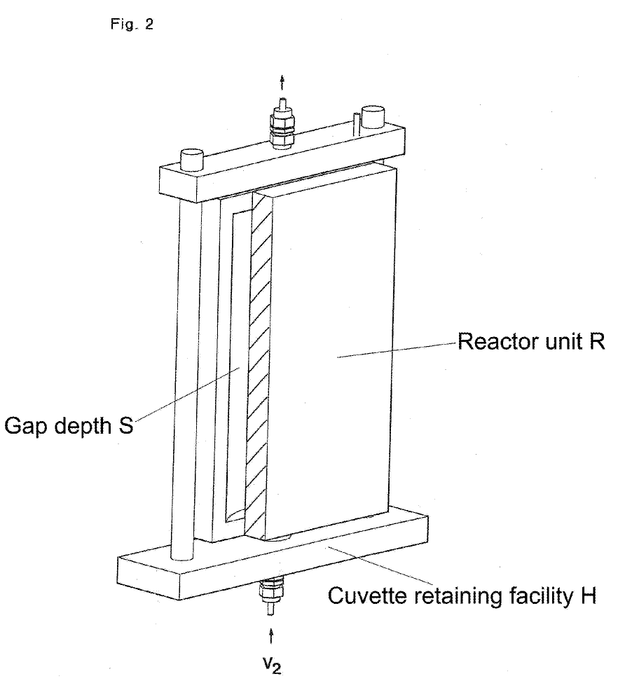

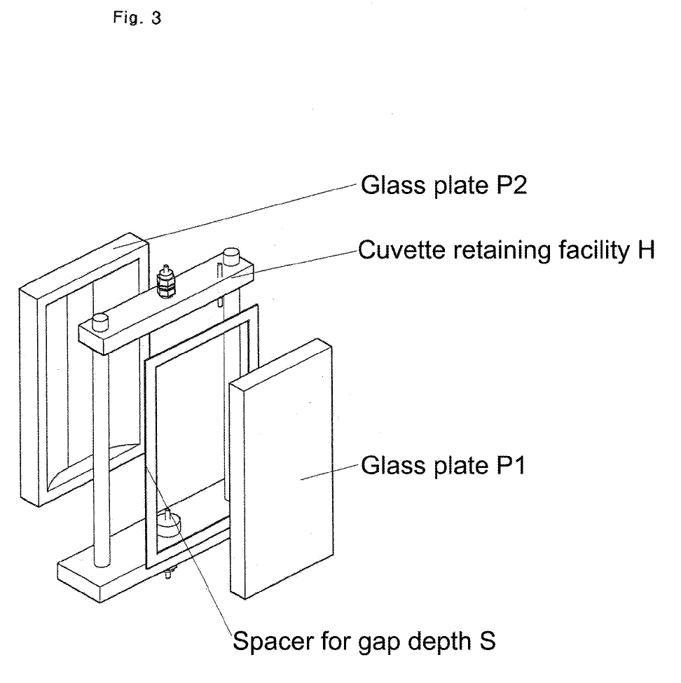

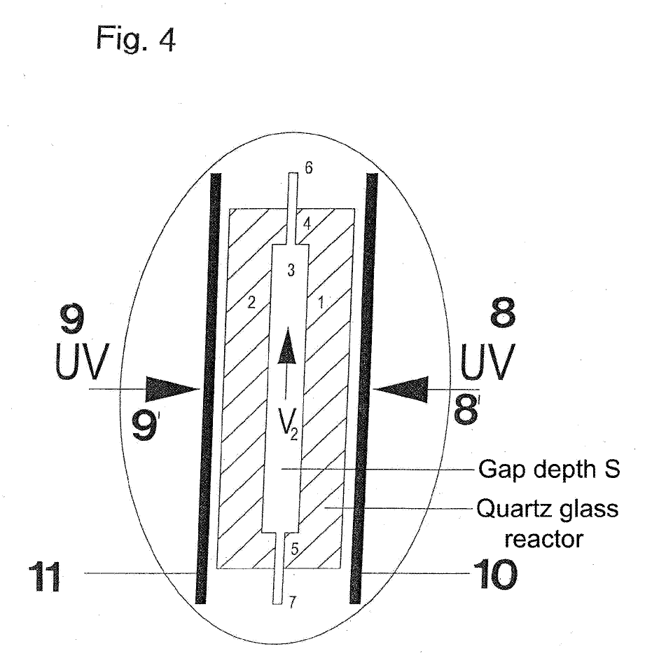

The diagrammatic representation of FIG. 4 shows, in the cross-section, the reactor unit R consisting of two quartz glass panes (P1 and P2 FIG. 3) 1, 2, connected in a flush manner with a spacer 4, 5 for the gap depth S and two spectral filters F. The incident UV light 8, 9 is represented by the two arrows. In the reactor, fluid stream flows at a flow rate of v2 in the direction of the black arrow in the cavity indicated by 3. It goes without saying that the fluid stream may pass through an entry zone before it reaches the irradiated zone.

What is claimed is:

1. A process for the production of 10-hydroxycamptothecin and/or of 7-alkyl 10-hydroxycamptothecin, comprising the steps of:
A providing a continuous fluid stream with a thickness d of a solution of camptothecin N-oxide and/or 7-alkyl camptothecin N-oxide; and
B. passing the stream past at least one source of light which produces radiation parallel to the thickness d, d being 10 μm to 1000 μm.

2. Process according to claim 1, wherein the fluid stream is provided in tubular or cuboid form with a length l, width b and thickness d.

3. Device for performing the process according to claim 1, said device comprising:
two transparent level quartz glass plates arranged in parallel at a distance d of 10 μm to 1000 μm,
two walls delimiting a space between the plates on two sides facing each other,
two apertures facing each other and not delimited by the walls, and
two sources of light arranged vertically to the plates,
such that their radiation penetrates into the space through the two plates,
wherein the plates are connected with each other in a firmly bonded manner.

4. Process according to claim 1, wherein d is 20 μm to 500 μm.

5. Process according to claim 4, wherein d is 40 μm to 100 μm.

6. Device according to claim 3, which further comprises filters for filtering out undesirable wavelength ranges, said filters being arranged between the sources of light and the quartz glass plates.

7. Process according to claim 1, wherein the source of light emits light in the range of 350 to 400 nm.

8. Process according to claim 1, wherein the source of light is at least one LED source with light of a wavelength of 375 or 385 nm.

9. Process according to claim 1, wherein the solvent consists of dimethyl formamide or a mixture of 1,4-dioxane and dimethyl formamide.

10. Process according to claim 9, wherein camptothecin N-oxide or 7-alkyl camptothecin N-oxide is present in the solution in a concentration of 0.1% to 3%.

11. Process for the production of 10-hydroxycamptothecin and/or of 7-alkyl 10-hydroxycamptothecin, comprising the steps of:
A providing a continuous fluid stream with a thickness d of a solution of camptothecin N-oxide and/or 7-alkyl camptothecin N-oxide; and
B. passing the stream past at least one source of light which produces radiation parallel to the thickness d, d being 10 μm to 1000 μm;
wherein said process is carried out simultaneously with several devices operated in parallel, each of said devices being a device according to claim 3.

12. Process for the production of 10-hydroxycamptothecin and/or of 7-alkyl 10-hydroxycamptothecin, comprising the steps of:
A providing a continuous fluid stream with a thickness d of a solution of camptothecin N-oxide and/or 7-alkyl camptothecin N-oxide; and
B. passing the stream past at least one source of light which produces radiation parallel to the thickness d, d being 10 μm to 1000 μm;
wherein said process is carried out with at least two devices connected in series, each of said devices being a device according to claim 3.
